(19) United States
(12) Patent Application Publication
(10) Pub. No.: US 2025/0236866 A1
(43) Pub. Date: Jul. 24, 2025

(54) EUGLOBULIN-BASED METHOD FOR DETERMINING THE BIOLOGICAL ACTIVITY OF DEFIBROTIDE

(71) Applicant: Gentium S.R.L., Villa Guardia(co) (IT)

(72) Inventors: Terenzio IGNONI, Solbiate (IT); Vijay KUMAR, Casnate (IT); Khalid ISLAM, Massagno (CH)

(21) Appl. No.: 19/173,652

(22) Filed: Apr. 8, 2025

Related U.S. Application Data
(63) Continuation of application No. 18/953,184, filed on Nov. 20, 2024.
Application No. 18/953,184 is a continuation of application No. 18/351,241, filed on Jul. 12, 2023, now abandoned.
Application No. 18/351,241 is a continuation of application No. 17/396,028, filed on Aug. 6, 2021, now Pat. No. 11,746,348.
Application No. 17/396,028 is a continuation of application No. 16/816,741, filed on Mar. 12, 2020, now Pat. No. 11,085,043.
Application No. 16/816,741 is a continuation of application No. 15/844,801, filed on Dec. 18, 2017, now abandoned.
Application No. 15/844,801 is a continuation of application No. 14/408,272, filed on Dec. 15, 2014, now Pat. No. 9,902,952.

(51) Int. Cl. C12N 15/11; A61K 31/711; C12Q 1/37; C12Q 1/56; G01N 33/86

(52) U.S. Cl. CPC C12N 15/11; A61K 31/711; C12Q 1/37; C12Q 1/56; G01N 33/86; C12N 2310/127

(57) ABSTRACT

It is disclosed a method for determining the biological activity of defibrotide, which comprises the steps of: a) bringing into contact defibrotide, mammalian euglobulin and a substrate specific for the plasmin which, by reaction with the plasmin, provides a measurable product; and b) measuring the amount of product formed at successive times, to thereby determine the biological activity of the defibrotide. Liquid defibrotide formulations are also disclosed, preferably water solutions, having a defined biological activity and, in particular, having an activity of 25 to 35 IU/mg of defibrotide, preferably from 27 to 32 IU/mg and, more preferably, from 28 to 32 IU/mg.

### CROSS-REFERENCE TO RELATED APPLICATIONS

This application is a continuation of U.S. application Ser. No. 18/953,184, filed Nov. 20, 2024, which is a continuation of U.S. application Ser. No. 18/351,241, filed Jul. 12, 2023, which is a continuation of U.S. application Ser. No. 17/396,028, filed Aug. 6, 2021, now U.S. Pat. No. 11,746,348, which is a continuation of U.S. application Ser. No. 16/816,741, filed Mar. 12, 2020, now U.S. Pat. No. 11,085,043, which is a continuation of U.S. application Ser. No. 15/844,801 filed Dec. 18, 2017, now abandoned, which is a continuation of U.S. application Ser. No. 14/408,272filed Dec. 15, 2014, now U.S. Pat. No. 9,902,952, and which is a 371 of international PCT/IT2012/000193, filed Jun. 22, 2012, each of which is herein incorporated by reference in its entirety.

### TECHNICAL FIELD OF THE INVENTION

The present invention relates to a method for determining the biological activity of defibrotide and, more especially, relates to an indirect enzymatic method for determining the biological activity of defibrotide.

### BACKGROUND OF THE INVENTION

Defibrotide (Merck Index, 1996, no. 2915) is a substance of natural origin which is obtained by extraction from animal organs and which is constituted by the sodium salt of polydeoxyribonucleotides having a low molecular weight. Defibrotide has been the subject of numerous pharmacological investigations which have suggested that it be applied in therapy as an anti-thrombotic agent (U.S. Pat. No. 3,829,567).

In addition, defibrotide has also been used successfully in the treatment of peripheral arteriopathies, in acute renal insufficiency (U.S. Pat. No. 4,694,134) or in acute myocardial ischaemia (U.S. Pat. No. 4,693,995).

Defibrotide is currently undergoing clinical trials to be used for the treatment and prevention of venous occlusive disease (VOD).

Like other biological substances which are obtained by extraction, defibrotide is also subject to a limited variability of composition which is typical of natural biopolymers. A classical example of this situation is provided by heparin whose variability from batch to batch in terms of chain length, molecular weight, composition, degree of sulphatation, etc. is well known. The consequence of this is that the same amounts by weight of defibrotide could in fact be non-equivalent from the point of view of a specific biological activity.

The process of extraction, isolation and purification cannot per se ensure absolute reproducibility of the product, precisely owing to its intrinsic biopolymeric nature.

However, if well controlled, it is possible to reduce this variability: for that purpose, studies have been made of standardized industrial processes for isolating defibrotide by extraction from organs, such as, for example, that described in U.S. Pat. No. 4,985,552.

The product obtained according to the above-mentioned process is characterized by the determination of some specific physico-chemical parameters, such as, for example, electrophoretic mobility, the coefficient of extinction, optical rotatory power and mass-average relative molecular mass. However, those parameters depend basically on the structure of defibrotide and are not capable of providing information on the biological activity thereof.

As far as we know, the only methods that have been reported to be used hitherto to evaluate the biological activity of defibrotide are the fibrin plate test and the thromboelastographic recording of the euglobulin lysis time [Prino G., Mantovani M., Niada R., Coccheri S., Butti A., Indagini preliminari sull'attivita fibrinolitica, nell'animale e nell'uomo, di una nuova sostanza presente in diversi organi animali, Simposio Internazionale: La ricerca scientifica nell'industria farmaceutica in Italia, Rome, 2-4 Oct. 1975-11 Farmaco, Ed. Prat.) (1969), 24,552-561] and the method based on plasmin disclosed in U.S. Pat. No 7,338,777, herein incorporated by reference.

However, the above-mentioned method thromboelastographic recording of the euglobulin lysis time is characterized by considerable experimental complexity, by unsatisfactory reproducibility and precision and, in the specific case of thromboelastographic recording, by a response linearity limited to very restricted concentration ranges.

As to the plasmin-based method, the enzymatic activity of plasmin is normally determined by various standard in vitro tests. One of the most commonly used methods is the determination by spectrophotometry or fluorimetry of the chromogenic or fluorogenic compounds that are freed by the action of plasmin on suitable substrates [Haemostasis, (1978), 7, 138-145]. Peptide substrates having the formula A₁-A₂-A₃-X are generally used in which A₁ and A₂ are amino acids that are predominantly non-polar, A₃ is lysine or arginine and X represents the measurable freed compound, for example para-nitroaniline (pNa) or 2-naphthylamine (NA) [Haemostasis, (1978), 7, 146-149]. In addition to the above-mentioned peptide substrates, success has been achieved using other, simpler, compounds, such as, for example, p-nitrobenzyl-p-toluenesulphonyl-L-arginine [Haemostasis, (1978), 7, 105-108].

The rate at which the compound X is released into the incubation medium is proportional to the activity (Units/mg) of plasmin present in the sample. The method disclosed in U.S. Pat. No 7,338,777 is thus based on the finding that, in the plasmin-evaluation tests described above, defibrotide increases the rate of release of compound X proportionally to its concentration.

However, such a method is conducted in TRIS buffer without any other plasma/serum activator/inhibitor. Therefore, the procedure does not reflect the physiological condition nor accurately simulates the mechanism of action of defibrotide in vivo.

Hitherto, therefore, no truly valid, precise and reproducible methods have been described and validated for determining the biological activity of defibrotide reflecting in an accurate way the mechanism of action of the product in a complex biological system (in vivo).

We have developed a simple and reliable method for determining the biological activity of defibrotide, which enables the samples obtained by extraction to be controlled and therefore enables medicinal preparations based on defibrotide to be standardized.

The method to which the present invention relates enables the specific biological activity of defibrotide to be determined in comparison with a reference standard with a high degree of precision and accuracy.

### BRIEF DESCRIPTION OF THE DRAWINGS

FIG. 1 is a plot showing the kinetics of release of pNA from the substrate S-2251, by means of mammalian euglobulin fraction which is activated and non-activated with defibrotide (concentration 0-50 μg/ml, 0-100 min).

FIG. 2 is a plot illustrating the sigmoid that arises in relation to a standard and test sample of defibrotide.

FIG. 3 is a plot showing the “absorbance versus time” of the standard preparations (ex.: S1_Ca, S1_Cb, S1_Cc) identify a suitable linear range (ex.: from 30 to 35 min).

### DESCRIPTION OF THE INVENTION

The present invention therefore relates to a method for determining the specific biological activity of samples of defibrotide, which method comprises the steps of:
a) bringing into contact defibrotide, euglobulin and a substrate specific for the plasmin which, by reaction with the plasmin, provides a measurable product and
b) measuring the amount of product formed at successive times.

The method of the invention is an indirect in vitro method for determining the activity of defibrotide, which is based on the functional interactions between defibrotide and euglobulin.

Euglobulin is that fraction of serum globulin that is insoluble in distilled water but soluble in saline solutions.

Euglobulin contains fibrinogen, PAI-1, tissue plasminogen activator (tPA), plasminogen, and to a lesser extent alpha 2-antiplasmin and also factor VIII.

The present inventors have surprisingly found that defibrotide catalyzes the hydrolysis of plasminogen into plasmin. Consequently, when defibrotide is incubated with euglobulin and a substrate specific for plasmin, such as a peptide of formula A1-A2-A3-X as disclosed by Haemostasis, (1978), 7, 138-149, herein incorporated by reference, the rate at which the compound X is released into the incubation medium increases proportionally to the concentration of defibrotide itself.

In other terms, defibrotide catalyzes the hydrolysis of plasminogen contained in euglobulin into plasmin; which plasmin enzymatically reacts with the substrate specific for plasmin, preferably a cromogenic substrate, to provide a measurable product.

The method of the present invention thus further comprises the steps of: c) determining the rate of release of the measurable product during the course of the enzymatic reaction of both a standard sample and a test sample of defibrotide; d) correlating, mathematically and/or graphically, the rate of release with the corresponding defibrotide concentration to obtain the biological activity of the test sample of defibrotide.

The defibrotide sample used for the determination according to the present invention is generally prepared by extraction from organs in accordance with known procedures, such as described, for example, in U.S. Pat. No. 4,985,552 which has already been mentioned and which is also herein incorporated by reference.

A batch of normal industrially manufactured defibrotide was chosen as the reference sample (standard) and was used to prepare the calibration curves in accordance with the method of the present invention.

In general, the present method provides precise and accurate measurements of defibrotide even in the presence of contaminants, such as, for example, RNA, heparin, degraded defibrotide (defibrotide from which purin or pyrimidine has been removed) or ethanol, provided they are in concentrations, generally less than 10% by weight, such as not to impair the system.

In addition to permitting the determination of defibrotide, the method also allows the determination of other biologically equivalent substances derived from defibrotide, such as, for example, deaminated defibrotide or, more simply, defibrotide denatured/degraded by combination of physicochemical conditions.

The present method is sufficiently sensitive to detect concentrations of defibrotide lower than or equal to 2.5 μg/ml (final concentration in the determination system) and, generally, expresses good correlation up to maximum concentration values higher than or equal to 1000 μg/ml.

The euglobulin used is generally any mammalian euglobulin fraction, such as, for example, bovine, porcine, rabbit ‘or human euglobulin, with a preference for human and bovine euglobuline.

However, although euglobulin fraction is the enzymatic system of choice, the use of other equivalent enzymatic systems, such as, for example, diluted plasma and serum (up to 1:10 with buffers), artificially created or isolated combinations of plasminogen, tPA, uPA, PAI-1&2 alpha 2 antiplasmin and the like enzymatic systems which are chemically and biologically related and have similar functionality, falls within the scope of the present invention.

In the method of the present invention, the substrate for the plasmin may be understood as being any substrate specific for plasmin which, under the conditions of the method, frees a detectable hydrolysis product X.

Depending on the nature of the detectable group X, alternative systems of detection commonly known to the person skilled in the art can be adopted equally well. Spectrophotometric or fluorimetric detection systems are particularly advantageous, especially spectrophotometric systems.

The substrates generally used are ones that are specific for plasminoge-plasmin assay. It is preferable to use peptides of the formula A₁-A₂-A₃-X, in which A₁ and A₂ are amino acids that are predominantly non-polar, A₃ is lysine or arginine and X is the detectable group. Examples of those substrates are Val-Leu-Lys-pNa, Val-Phe-Lys-pNa or pyroGlu-Phe-Lys-pNa in which the group X detectable by spectrophotometry is para-nitroaniline (pNA). Other suitable substrates, for example Val-Gly-Arg-2NA, contain 2-naphthylamine, which is measurable by fluorimetry. A particularly preferred substrate is the compound H-D-Valyl-L-Leucyl-L-Lysine-p-nitroaniline (H-D-Val-Leu-Lys-pNA).

The specific substrates used for determining defibrotide activity in euglobulin fraction are generally commercially available.

The determination method of the present invention is carried out by placing defibrotide sample in euglobulin solution, at a specific pH and molarity.

In particular, euglobulin fractions obtained from mammalian plasma are reconstituted dissolving and diluting the euglobulin to the original volume of the generating plasma with saline buffer (ex.: the quantity of euglobulin fraction obtained from 10 mL plasma are dissolved and reconstituted to 10 mL with saline buffer at pH between 7 and 8).

However, as regards of the substrate concentrations of from 0.3 to 4 mM, preferably from 2.5 to 3.5 mM and advantageously of 3 mM, are generally used in the case of a chromogenic substrate, while concentrations of from 0.05 to 0.15 mM are used in the case of a fluorogenic substrate.

The determination method of the invention, like other enzymatic methods, is sensitive to the pH of the medium.

In fact, it cannot generally be applied at extreme pH values where the enzymatic system would be inactivated.

It is also preferable for the pH of the medium not to undergo variation at any time during the period when measurements are being taken, and therefore euglobuline fraction is reconstituted with buffer systems selected from those normally used for biological tests. Suitable buffer systems may be, for example, phosphate buffer, citrate buffer or tris (hydroxymethyl) aminomethane hydrochloride and sodium chloride (TRIS-NaCl) buffer. The reconstitution of the euglobuline fraction is preferably carried out with TRIS-NaCl.

In the present method it is usually preferred to maintain the pH of the medium in a range of approximately from 7 to 8, more preferably at approximately 7.4-7.6.

In addition, it is preferred to maintain the concentration of the buffer system in a range of from 10 to 200 mM, preferably at approximately 50 mM. More specifically for the TRIS-NaCl the concentrations should be 50 mM for TRIS and 150 mM for sodium chloride.

The method of the invention for determining defibrotide biological activity, defibrotide sample solutions is diluted directly into euglobulin fraction, then the chromogenic or fluorogenic substrate is added. In particular, in order to enable the measurements it is preferable to preliminary dilute/dissolve defibrotide in TRIS-NaCl buffer in order to obtain a mother stock solution of both, sample and standard. From the mother stock solutions the sample and the standard are diluted, by serial dilution, into defined volume of euglobulin fraction till the analytical concentration range which is about 1 to 1000 μg/mL of defibrotide.

An important parameter in the present method of determination is the temperature. It is preferable for the same temperature to be maintained throughout the entire duration of the measurements and for all of the samples determined, both as regards the construction of the reference curves and during the measuring stage. To that end, it is preferable to use temperature controlled apparatus and also, where necessary, it is possible to proceed with several sets of measurements, changing the position of the samples appropriately in order to ensure that the system has maximum thermal homogeneity.

Generally, this method of determination is applied in a temperature range of, for example, from 25 to 40° C., preferably from 35 to 39° C., and even more preferably at 37° C.

According to the present invention, measurement of the concentration of compound X released in the medium by the action of defibrotide starts when all of the reagents have been added and continues for a predetermined time and at a predetermined frequency as a function of the chemical nature of X and of the detection system.

Similarly to other methods of biological determination, the method of the invention also provides for a calibration stage and a measuring stage which are preferably carried out in the same microplateplate in order to reduce as far as possible the incidence of experimental variability.

The calibration stage comprises the acquisition of the absorbance data relating to the samples at known increasing concentrations of defibrotide (standard), the statistical reprocessing of those data and the extrapolation of calibration curves, which express the correlation between the increase in the rate of the enzymatic reaction of the invention and the concentration of defibrotide present in the euglobulin fraction. In the measuring stage, owing to the correlation obtained in the calibration stage, it is possible to determine the unknown biological activities of samples of defibrotide on the basis of the absorbance values measured and processed under the same conditions.

In more detail, the experimental protocol generally provides for the preparation of several samples, both standard and unknown, at various known concentrations of defibrotide. The defibrotide samples are prepared by progressive dilution of the mother solutions in accordance with a predetermined dilution factor.

In the present method, it is preferred to prepare at least 5 concentrations of the standard and 5 concentrations of the sample to be tested, preparing at least 4 replicates for each concentration of the standard and, similarly, for each concentration of the test sample, generally for successive 1:2 dilutions of mother solutions.

Both the standard and test-sample concentrations of defibrotide are generally from 0.1 to 1000 μg/ml.

The concentrations of the test sample are preferably of the same order of magnitude as the concentrations of the standard.

In accordance with the above illustration, the measurements for each concentration are preferably carried out on 1 microplates where the position of each sample, the standard and the test sample, respectively, at corresponding concentration is preferably alternated. According to this scheme for the arrangement of the samples, which is explained in more detail in the experimental part, for each concentration of both standard and test-sample defibrotide, at least 4 absorbance values are measured for each time.

The set of measurements described above are taken at predetermined times, that is to say, first of all at time to, that is to say, when all of the components have been added, before the enzymatic reaction of the invention has started, and subsequently at precise intervals and for a period of time sufficient to acquire the necessary data.

Preferably, the absorbance measurements are continued up to a maximum of 90 minutes, with readings taken every 1-10 minutes. More advantageously, the readings are taken every minute. The photometric absorbance readings are performed at a wavelength which depends on the nature of the detectable group X freed in the course of the enzymatic hydrolytic reaction. In the specific case in which X is p-NA, the absorbance is measured at 405 nm.

The absorbance readings of the standard and unknown defibrotide samples, known as raw data, generally originate directly from the same apparatus that provides for the reading operation; they are tabulated in such a manner that an absorbance value is expressed for each time and well.

The raw data are then processed, using, for example, the Spread Sheet—Microsoft Excel®. This first processing operation leads to the calculation of the average absorbance and of the associated standard deviation, at each time and for each set of readings, each set comprising at least 4 replicates for each concentration of both standard and test-sample defibrotide.

Further statistical processing of the data is carried out with commercially available software for biological assay determination as described by Finney D J, Statistical Method in Biological Assay, 2nd ed. Ch. Griffin, London and relevant Pharmacopoeias.

To be more precise, according to the present invention, defibrotide biological assay determination can be performed using parallel line model, slope ration model and four-parameter logistic curve models as defined, for example, by the relevant European Pharmacopoeia General text 5.3, Statistical Analysis.

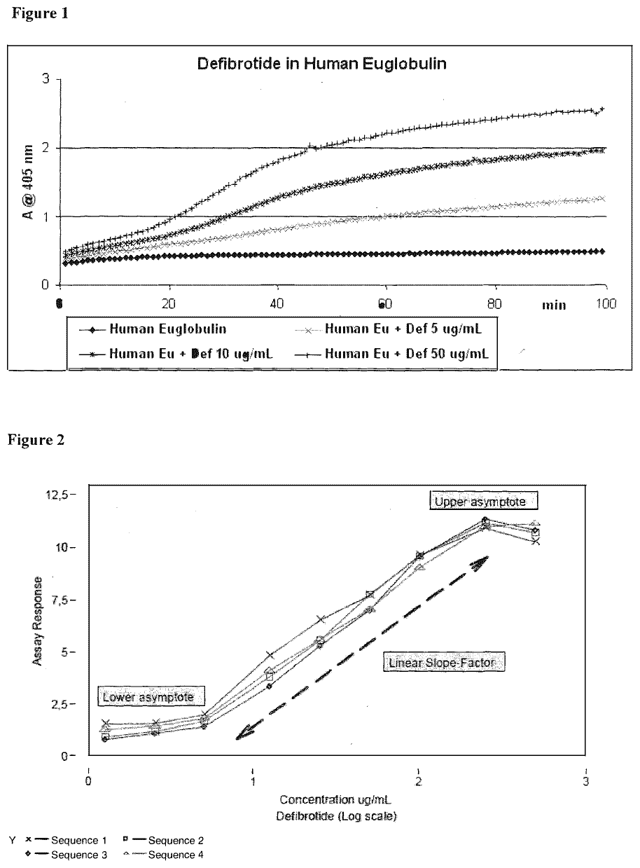

As illustrated in FIG. 1, by placing the time on the abscissa and the absorbance on the ordinate, straight lines will be obtained whose slope “b” will be proportional to the rate of enzymatic reaction: by increasing the concentration of defibrotide, the rate of hydrolysis and, proportionally, the value of “b” will increase. Finally, the slope values, calculated as described above for each set of replicates of standard defibrotide and test-sample defibrotide, are correlated with the concentration of defibrotide to which they relate. Suitable mathematical transformation of the abscissa (i.e. log of defibrotide concentration) can be used in lieu of the real value.

Graphically, that correlation gives rise to a sigmoid for the standard and a sigmoid for the test sample (FIG. 2); the central portions of the sigmoid have two straight lines which are generally parallel and the distance between which is a function of the difference in biological activity between the test sample and the standard. This interval is used for potency determination using the parallel line model as described by Finney D J, Statistical Method in Biological Assay, 2nd ed. Ch. Griffin, London.

For a more specific determination, the four-parameter logistic curve models is used and in this case the entire sigmoid curve of both, sample and standard, is used for the calculation of the relative biological potency of the sample.

In a preferred embodiment of the present invention, the standard solutions and the solutions of the samples of defibrotide to be determined are introduced into the respective wells of the microplate. The euglobulin fraction are prepared at the moment of use and it is the dilution media of defibrotide stock solution. Finally, the solution containing the chromogenic substrate is added. The microplate is then placed in the thermostated reader and, after rapid agitation, readings of the system's absorbance are taken at predetermined intervals and for the predetermined period of time. The raw data obtained are then processed, thus determining the unknown activities of the defibrotide samples.

As it shall be appreciated from the following examples, the method according to the present invention allows to obtain liquid defibrotide formulations, preferably water solutions, having a defined biological activity and, in particular, having an activity of 25 to 35 IU/mg of defibrotide, preferably from 27.5 to 32.5 IU/mg and, more preferably, from 28 to 32 IU/mg.

Liquid defibrotide formulations are preferably marketed in form of containers, more preferably vials, containing 200 mg of defibrotide in 2.5 ml of buffered water solution (preferably at a pH from 6.5 to 8.5, more preferably from 7 to 8), to be diluted before use; consequently, when the biological activity is assessed with the method of the present invention, each container presents a defibrotide activity of 5000 to 7000 IU, preferably 5500 to 6500 IU, more preferably 5600 to 6400.

Those and other aspects of the invention will be better illustrated in the following examples which are not, however, to be regarded as limiting the invention.

### EXAMPLES

The following materials were used in the examples given here:

### Apparatus

| Main features: | Microplate reader for UV-Vis absorbance | | |
|---|---|---|---|
| | determination equipped with thermostatic | | |
| | chamber and absorbance filter at 405 nm. | | |
| Detection | Kinetic Absorbance determination at 405 nm | | |
| Plate Type | 96-well clear for UV-Vis Determination | | |
| Chamber | 37° C. | Total Absorbance | 45-60 min |
| Temperature | | recording time | |
| Absorbance | About 1 × min | | |
| recording frequency | | | |

### Programs & Software

Microsoft Excel® (Microsoft Corporation, Redmond, Wash., USA)

### Substances

Defibrotide (Gentium)
Chromogenic substrate S-2251 (Chromogenix Instrumentation Laboratory S.p.A., Milan, Italy)
Tris (hydroxymethyl) aminomethane (TRIS)-NaCl, (Sigma-Aldrich, Milan, Italy)
1N HCl (Carlo Erba reagenti, Milan, Italy)
1N NaOH (Carlo Erba reagenti, Milan, Italy)
Bovine Plasma (Tebu Bio Italia, Magenta (MI), Italy)
Glacial Acetic Acid (Carlo Erba reagenti, Milan, Italy)

### Solutions

TRIS-NaCl (1 L Preparation): Into a 1 L beaker quantitatively transfer 6.06 g of TRIS-HCl and 2.2 g of NaCl. Dissolve in 500 mL of purified water and adjust the pH to 7.4 with about 40 mL of HCl 1N. Quantitatively transfer the solution into a volumetric flask of 1 L and dilute the solution to volume with purified water. Store the solution into refrigerator (2-8° C.)
Chromogenic Substrate S2251 (CAS 63589-93-5) 3 mM (15.2 mL Preparation): Dissolve about 25 mg of chromogenic substrate with 15.2 mL of purified water. Store the solution into refrigerator (2-8° C.)
Bovine euglobulins (10 mL Preparation). In a container with a minimum capacity of 300 mL introduce 240 mL of ice-cooled purified water and under stirring add 10 mL bovine plasma. Adjust the pH to 5.3 +0.1 with acetic acid 1%. Allow to settle at 2-8° C. for 1 to 16 hours. Remove the clear supernatant solution by siphoning and collect the precipitate by centrifugation at 2.800 rpm for 5 minutes and 4° C. Suspend the precipitate dispersing mechanically (e.g.,: by means of a laboratory glass rod) in 5 mL of ice-cooled purified water, shake for about 5 min and collect the precipitate by centrifugation at 2.800 rpm for 5 minutes and 4° C. Disperse the precipitate mechanically into about 10 mL of TRIS-NaCl; to facilitate the dissolution of the precipitate crush the particles of the precipitate with a suitable instrument (es.: laboratory glass rod). Store the obtained suspension at 2-8° C. for not less than 1 hour and not more than 6 hours before its use.

### Standard and Sample Defibrotide Solutions

### Reference Stock Solution

Into 20 mL volumetric flask quantitatively transfer about 100 mg of defibrotide reference standard accurately weighed. Dissolve the powder with about 10 mL TRIS-NaCl and bring the volume with the same solvent. Dilute 1:4 the obtained solution with TRIS-NaCl in order to obtain a defibrotide RS solution of about 1.25 mg/mL.

### Sample Stock Solution

Into 20 mL volumetric flask quantitatively transfer about 100 mg of defibrotide sample accurately weighed. Dissolve the powder with about 10 mL TRIS-NaCl and bring the volume with the same solvent. Dilute 1:4 the obtained solution with TRIS-NaCl in order to obtain a defibrotide sample solution of about 1.25 mg/mL.

### Reference and Sample Solutions Preparation

a) Defibrotide 125 μg/mL: dilute 1:10 defibrotide stock solution (Reference and Sample) with TRIS NaCl (corresponding to 50 μg/mL into the plate well). Into an eppendorf tube quantitatively transfer 500 μL of the prepared solution and mix with 500 μL of euglobulin. Close the tube and store in ice-cooled water.
b) Defibrotide 62.5 μg/mL: dilute 1:2 solution (a) with TRIS NaCl (corresponding to 25 μg/mL into the plate well), Into an eppendorf tube quantitatively transfer 500 μL of the prepared solution and mix with 500 μL of euglobulin. Close the tube and store in ice-cooled water.
c) Defibrotide 31.25 μg/mL: dilute 1:2 solution (b) with TRIS NaCl (corresponding to 12.5 μg/mL into the plate well). Into an eppendorf tube quantitatively transfer 500 μL of the prepared solution and mix with 500 μL of euglobulin. Close the tube and store in ice-cooled water.
d) Defibrotide 12.5 μg/mL: dilute 1:2.5 solution (d) with TRIS NaCl (corresponding to 5 μg/mL into the plate well). Into an eppendorf tube quantitatively transfer 500 μL of the prepared solution and mix with 500 μL of euglobulin. Close the tube and store in ice-cooled water.

### Blank Solution

Mix 1 volume of euglobulins with 1 volume of TRIS NaCl solution (ex.: 500 μL+500 μL)

### Plate Deposition

According to the proposed deposition scheme (see Table 1 below) add in each well of the plate 200 μL of standard or sample or blank solution. Note different deposition scheme can be used according to the availability and configuration of automatic pipettes. However not less than 4 depositions for each reference and sample solution must be used for the assay.

Immediately before incubation of the plate into the microplate reader add in each well 50 uL of Chromogenic substrate.

TABLE 1

| | 1 | 2 | 3 | 4 | 5 | 6 | 7 | 8 | 9 | 10 | 11 | 12 |
|---|---|---|---|---|---|---|---|---|---|---|---|---|
| A | — | BLK | S1_Ca | S1_Cb | S1_Cc | S1_Cd | — | — | — | — | — | — |
| B | — | BLK | S2_Ca | S2_Cb | S2_Cc | S2_Cd | — | — | — | — | — | — |
| C | — | BLK | S3_Ca | S3_Cb | S3_Cc | S3_Cd | — | — | — | — | — | — |
| D | — | BLK | S4_Ca | S4_Cb | S4_Cc | S4_Cd | — | — | — | — | — | — |
| E | — | BLK | U1_Ca | U1_Cb | U1_Cc | U1_Cd | — | — | — | — | — | — |
| F | — | BLK | U2_Ca | U2_Cb | U2_Cc | U2_Cd | — | — | — | — | — | — |
| G | — | BLK | U3_Ca | U3_Cb | U3_Cc | U3_Cd | — | — | — | — | — | — |
| H | — | BLK | U4_Ca | U4_Cb | U4_Cc | U4_Cd | — | — | — | — | — | — |

S1, S2, S3, S4: Reference Solution deposition 1, deposition 2, deposition 3, deposition 4, U1, U2, U2, U5: Sample solution deposition 1, deposition 2 deposition 3, deposition 4, Ca, Cb, Cc etc.: Defibrotide reference and sample concentration a, b, c etc.

BLK Blank Solution

Calculation and Results

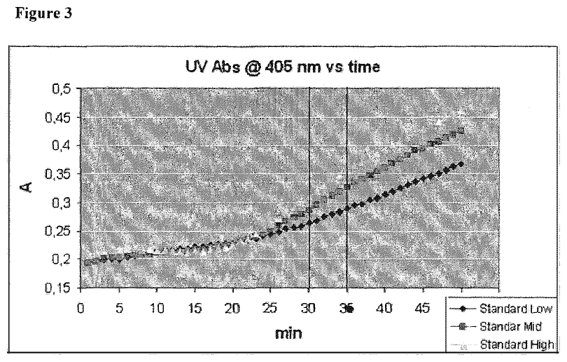

From the kinetic plot “absorbance versus time” of the standard preparations (ex.: S1_Ca, S1_Cb, S1_Cc) identify a suitable linear range (ex.: from 30 to 35 min, see FIG. 3).

Identification of the linear kinetic range (A@405 nm vs time).

Calculate for each preparation of the standard and of the sample the response of the assay (Slope) in the pre-defined time range as follow.

$$Sample \& Standard Response = \frac{{A}_{b} - {A}_{a}}{{T}_{b} - {T}_{a}} \times 1000$$

### Where:

Aa is the Absorbance values at Time Ta (30 min from the plot above)
Ab is the Absorbance value at Time Tb (35 min from the plot above)

Report the obtained value in a tabular format as reported in table 2.

TABLE 2

| Concentration | | | | | | | | |
|---|---|---|---|---|---|---|---|---|
| Level | Standard Preparation | | | | Sample Preparation | | | |
| [ug/mL] | S1 | S2 | S3 | S4 | U1 | U2 | U3 | U4 |
| 5 | S1_Cd | S2_Cd | S3_Cd | S4_Cd | U1_Cd | U2_Cd | U3_Cd | U4_Cd |
| 12.5 | S1_Cc | S2_Cc | S3_Cc | S4_Cc | U1_Cc | U2_Cc | U3_Cc | U4_Cc |
| 25 | S1_Cb | S2_Cb | S3_Cb | S4_Cb | U1_Cb | U2_Cb | U3_Cb | U4_Cb |
| 50 | S1_Ca | S2_Ca | S3_Ca | S4_Ca | U1_Ca | U2_Ca | U3_Ca | U4_Ca |

Plot the responses for the substance to be examined and for the standard against the logarithms of the concentration and calculate the activity of the substance to be examined using the parallel line model as defined by the relevant 5.3.2 chapter of the Ph. Eur Current edition.

Not less than 3 consecutive serial dilutions of the reference and of the sample should be used (e.g., defibrotide concentration 5 μg/mL, 12.5 μg/mL, 25 μg/mL, 50 μg/mL, or 5 μg/mL, 12.5 μg/mL, 25 μg/mL, or 12.5 μg/mL, 25 μg/mL, 50 μg/mL).

### Analysis of the Variance

The analysis of the variance is performed according to the section 5.3.2.3 of the Ph. Eur. current edition and Finney DJ (1964) Statistical Method in Biological Assay 2nd ed.

### Test for Validity

1) The linear regression terms is significant, i.e. the calculated probability is less than 0.05. If this criterion is not met, it is not possible to calculate 95% C.I.
2) The Term of non-parallelism is not significant, i.e. the calculated probability is less than 0.05.
3) The term for non linearity is not significant, i.e. the calculated probability is less than 0.05.

### Acceptance Criteria

4) The estimated potency is not less than 90% and not more than 111% of the stated potency.
5) The confidence limits (P-0.95) of the estimated potency are not less than 80% and not more than 125% of the stated potency

### Calculation

$$Sample Potency ( UI / mg ) = \frac{R \times Std Potency ( UL / mg ) \times Conc . Std ( mg / mL )}{Conc . Sample ( mg / mL )}$$

where:
R: Result of sample obtained by the parallel line model analysis
Std Potency: Stated potency of the Standard (UI/mg on dry substance)
Conc. Std: Concentration of the Standard (mg/mL on dry substance)
Conc. Sample: Concentration of the Sample (mg/mL on dry substance)

### Assay Applied to Defibrotide Formulations

The above-disclosed assay has been used to determine the biological activity of liquid formulations containing 200 mg of defibrotide in 2.5 ml (80 mg per ml) and having the quali-quantitative composition reported in table 3.

TABLE 3

| | Reference to | | | | |
|---|---|---|---|---|---|
| | Standard | | 200 mg | Concentration | |
| Component | Quality | Function | Injection | per mL | |
| Defibrotide | In-house | Drug | 200.00 mg | 80.00 | mg |
| | standard | Substance | | | |
| Sodium Citrate, | USP-EP | Buffer | 25.00 mg | 10.00 | mg |
| Dihydrate | | | | | |
| Water-for- | USP-EP | Solvent/ | q.s. to | 1.00 | mL |
| injection | | vehicle | 2.5 mL | | |

| | | | | |
|---|---|---|---|---|
| Sodium | NF-EP | pH | q.s. to | — |
| hydroxide | | adjustment | 6.8-7.8 | |
| 1M or | | | | |
| hydrochloric | | | | |
| acid 1M | | | | |
| Nitrogen | NF-EP | Inert gas | q.s. | — |
| | | to displace | | |
| | | the air | | |

The results are reported in table 4.

TABLE 4

| | Potency | Potency |
|---|---|---|
| Batch | IU/mg | IU/container of 200 mg |
| 688 | 31 | 6200 |
| 738 | 34 | 6800 |
| 785 | 32 | 6400 |
| 836 | 30 | 6000 |
| 0406 | 31 | 6200 |
| DS0617 | 35 | 7000 |
| DV0502* | * | Not available |
| DV0601 | 35 | 7000 |
| 1070010073 | 32 | 6400 |
| 1080010016 | 32 | 6400 |
| 1080020018 | 31 | 6200 |
| 1080030021 | 31 | 6200 |
| 1080040110 | 32 | 6400 |
| 1080050114 | 35 | 7000 |
| 1080060117 | 34 | 6800 |
| Mean | 33 | |
| Min | 30 | |
| Max | 35 | |
| RSD (%) | 5, 4 | |

*Used as Reference Standard

1. A defibrotide formulation comprising defibrotide, sodium citrate, and water for injection, having a potency of 25 to 35 IU/mg, and a concentration of at least 80 mg/mL, wherein the formulation is free of methyl p-hydroxybenzoate and propyl p-hydroxybenzoate, wherein the defibrotide potency is determined by a method comprising the steps of:
a) bringing into contact defibrotide, a biological composition comprising plasminogen, and a substrate specific for plasmin which, by reaction with plasmin, provides a measurable product; and
b) measuring the amount of product formed at successive times, to thereby determine the potency of the defibrotide.

2. The defibrotide formulation of claim 1, wherein the formulation has a potency of 27.5 to 32.5 IU/mg.

3. The defibrotide formulation of claim 2, wherein the formulation has a potency of 28 to 32 IU/mg.

4. The defibrotide formulation of claim 1, wherein the formulation is a water solution.

5. The defibrotide formulation of claim 4, wherein the formulation has a pH of from 6.5 to 8.5.

6. The defibrotide formulation of claim 5, wherein the formulation has a pH of from 7 to 8.

7. The defibrotide formulation of claim 5, wherein the formulation additionally comprises a pH adjusting agent.

8. The defibrotide formulation of claim 7, wherein the pH adjusting agent is an acid and/or a base.

9. The defibrotide formulation of claim 7, wherein the pH adjusting agent is hydrochloric acid, sodium hydroxide, or a combination thereof.

10. A defibrotide formulation comprising defibrotide, sodium citrate, and water for injection, having a potency of 25 to 35 IU/mg, and a concentration of at least 80 mg/mL, wherein the formulation is free of parabens, wherein the defibrotide potency is determined by a method comprising the steps of:
a) bringing into contact defibrotide, a biological composition comprising plasminogen, and a substrate specific for plasmin which, by reaction with plasmin, provides a measurable product; and
b) measuring the amount of product formed at successive times, to thereby determine the potency of the defibrotide.

11. The defibrotide formulation of claim 10, wherein the formulation has a potency of 27.5 to 32.5 IU/mg.

12. The defibrotide formulation of claim 11, wherein the formulation has a potency of 28 to 32 IU/mg.

13. The defibrotide formulation of claim 10, wherein the formulation is a water solution.

14. The defibrotide formulation of claim 13, wherein the formulation has a pH of from 6.5 to 8.5.

15. The defibrotide formulation of claim 14, wherein the formulation has a pH of from 7 to 8.

16. The defibrotide formulation of claim 14, wherein the formulation additionally comprises a pH adjusting agent.

17. The defibrotide formulation of claim 16, wherein the pH adjusting agent is an acid and/or a base.

18. The defibrotide formulation of claim 16, wherein the pH adjusting agent is hydrochloric acid, sodium hydroxide, or a combination thereof.

19. A defibrotide formulation comprising defibrotide, sodium citrate, and water for injection, having a potency of 25 to 35 IU/mg, and a concentration of at least 80 mg/mL, wherein the formulation is free of preservatives, wherein the defibrotide potency is determined by a method comprising the steps of:
a) bringing into contact defibrotide, a biological composition comprising plasminogen, and a substrate specific for plasmin which, by reaction with plasmin, provides a measurable product; and
b) measuring the amount of product formed at successive times, to thereby determine the potency of the defibrotide.

20. The defibrotide formulation of claim 19, wherein the formulation has a potency of 27.5 to 32.5 IU/mg.

21. The defibrotide formulation of claim 20, wherein the formulation has a potency of 28 to 32 IU/mg.

22. The defibrotide formulation of claim 19, wherein the formulation is a water solution.

23. The defibrotide formulation of claim 22, wherein the formulation has a pH of from 6.5 to 8.5.

24. The defibrotide formulation of claim 23, wherein the formulation has a pH of from 7 to 8.

25. The defibrotide formulation of claim 23, wherein the formulation additionally comprises a pH adjusting agent.

26. The defibrotide formulation of claim 25, wherein the pH adjusting agent is an acid and/or a base.

27. The defibrotide formulation of claim 25, wherein the pH adjusting agent is hydrochloric acid, sodium hydroxide, or a combination thereof.
